(19) United States
(12) Patent Application Publication
(10) Pub. No.: US 2018/0228647 A1
(43) Pub. Date: Aug. 16, 2018

(54) System and Method for Opthalmic Surgical Procedures

(71) Applicants: Luis Jose Escaf, Barranquilla (CO); Luis Carlos Escaf, Barranquilla (CO)

(72) Inventors: Luis Jose Escaf, Barranquilla (CO); Luis Carlos Escaf, Barranquilla (CO)

(21) Appl. No.: 15/429,863

(22) Filed: Feb. 10, 2017

(51) Int. Cl. A61F 9/00; A61F 9/007; A61M 5/172; A61B 3/16

(52) U.S. Cl. CPC A61F 9/0008; A61F 9/00745; A61M 5/1723; A61M 2210/0612; A61M 2005/1726; A61B 2217/005; A61B 2217/007; A61B 3/16

(57) ABSTRACT

The present invention discloses a system and method for delivery viscoelastic, materials into the eye based on the intraocular pressure of the eye, thereby reducing the distraction of the surgeon from other activities involved in the surgery, and increasing the consistency and accuracy of the use of viscoelastic materials during surgery, from surgeon to surgeon.

### BACKGROUND

The present invention generally relates to medical devices and procedures, and particularly to systems and methods used during surgery for the treatment of cataracts.

A cataract is a clouding of the lens inside the eye, causing vision degradation or loss that cannot be corrected with glasses, contact lenses or corneal refractive surgery like laser in-situ keratomileusis (LASIK). There are, however, surgical procedures for cataracts. In cataract surgery, the lens inside your eye that has become cloudy is removed and replaced with an artificial lens (called an intraocular lens, or IOL).

One procedure fir removing the cloudy lens, called phacoemulsification or “phaco,” involves breaking up the cloudy lens into small pieces, which are then gently removed from the eye with suction. After all the remnants of the cloudy lens have been removed from the eye, the cataract surgeon inserts a clear intraocular lens, positioning it securely behind the iris and pupil, typically in the same location your natural lens occupied.

To perform the phaco procedure, the surgeon typically uses an ultrasonic surgical device consisting of an ultrasonically driven handpiece, an attached cutting tip, an irrigating sleeve and an electronic control console. The handpiece assembly is connected to the control console through an electrical cable and a flexible tube. During the phaco procedure, the control console varies the power level transmitted by the handpiece to the cutting tip, and the flexible tubing is used to supply irrigation fluid to and draw aspiration from the eye through the handpiece. The operative part of the handpiece is a centrally located, hollow resonating bar or horn directly attached to piezoelectric crystals. The crystals supply the required ultrasonic vibration needed to drive both the horn and the attached cutting tip during phacoemulsification and are controlled by the console. The crystal/horn assembly is suspended with a hollow body or shell of the handpiece by flexible mountings. The handpiece body terminates in a reduced diameter portion or nosecone at the body's distal end. The nosecone is externally threaded to accept the irrigation sleeve. Similarly, the horn bore is internally threaded at its distal end to receive the external threads of the cutting tip. The irrigation sleeve also has an internally threaded bore that is screwed onto the external threads of the nosecone. The cutting tip is adjusted so that the tip extends only a predetermined amount past the open end of the irrigation sleeve.

In use, the ends of the cutting tip and irrigating sleeve are typically inserted into a small incision in the cornea. The cutting tip is ultrasonically vibrated by the crystal-driven ultrasonic horn, thereby emulsifying the selected lens tissue. The hollow bore of the cutting tip communicates with the bore in the horn that in turn communicates with the aspiration line from the handpiece to the console. A reduced pressure or vacuum, source in the console draws or aspirates the emulsified tissue from the eye through the open end of the cutting tip and horn bores and the aspiration line and into a collection device. The aspiration of emulsified tissue is aided by a saline flushing solution that is injected into the surgical site through the small annular gap between the inside surface of the irrigating sleeve and the cutting tip.

It has been found that using such an ultrasonically driven handpiece in phaco procedures can cause damage to the eye. Particularly, it has been found that the ultrasonic energy used to emulsify the cloudy tissue of the lens can cause damage to the endothelial cells of the cornea. Cortical endothelial help balance the flow of fluid into and out of the cornea, which helps the cornea remain transparent and therefore very important to clear vision. It is believed that the ultrasound energy used during phacoemulsification causes endothelial cell loss and/or damage during surgery and it also causes endothelial cell loss at a higher than normal rate for 10 years following the surgery. Since the endothelial cells of the cornea do not reproduce when damaged, endothelial cell loss can produce a range of different problems, from corneal edema to corneal descompensation or Bullous Keratopathy, in which the cornea loses its transparency resulting in the loss of visual acuity.

A solution to this problem has been to inject viscoelastic materials into the eye to maintain the stability of the anterior chamber of the eye, thus preventing its collapse during the procedure, and also to protect the corneal endothelium. The viscoelastic materials, sometimes referred to as ophthalmic viscoelastic devices (OVDs), have a viscous gel-like composition, which is used to coat the chambers of the eye in order to protect sensitive tissue in particular, endothelial cells, from trauma caused by the ultrasonic energy used in a phaco procedure. There are many such viscoelastic materials available today including, for example, Viscoat® and Healon®. A description of the types of viscoelastic materials utilized and methods utilized is given in U.S. Pat. No. 5,358,473, which is incorporated herein by reference in order to provide disclosure for the types of viscoelastic materials and the procedures commonly used to administer them. It has been found that the use of viscoelastic materials during a phaco procedure can reduced the incidence of endothelial cell damage both during and after the cataract surgery.

The common approach to using viscoelastic materials during a phaco procedure is to inject the fluids into the eye chambers by means of a hand held syringe or cannula. Because the flow characteristics and viscosity of the viscoelastic materials vary to some degree depending on factors such as the composition of the material, the temperature of the material, and the geometry of the injection apparatus, a manually-operated syringe is commonly used to enable direct physician control of the injection rate of the viscoelastic material into the eye.

The viscoelastic material is typically manually injected into the eye at the beginning of the surgical procedure and then removed from the eye to begin the phaco procedure. However, during surgery, the constant irrigation/aspiration and use of ultrasonic power to emulsify the target cloudy tissue tends to wash the viscoelastic material away from the sensitive tissue (e.g., endothelium) in the eye. This causes the endothelium to be more susceptible to short and long-term damage as described above.

In order to prevent such damage to the endothelium, the surgeon needs to add or direct an assistant to periodically add more viscoelastic material to the eye during the phaco procedure. This requires the surgeon to visually monitor the amount of viscoelastic material physically present in the eye during the surgery and actually stop the surgery so that a syringe or needle can be re-inserted into the eye whenever he/she decides more viscoelastic material needs to be added. The distraction from the surgical procedure to observe the viscoelastic material and the potentially repeated interruptions to re-insert a viscoelastic syringe or needle into the eye is highly undesirable, and can increase the risk of infection in the eye. In addition, since the decision as to whether more viscoelastic material should be added depends solely on the judgment and experience of the surgeon and/or the surgeon's assistant, such phaco procedures are subject to error and inconsistency from surgery to surgery and surgeon to surgeon.

One method for addressing the problems associated with repeated re-insertions of a syringe or needle into the eye each time additional viscoelastic materials need to be injected is disclosed in U.S. Pat. No. 6,254,587 (the '587 patent), which is incorporated herein by reference. The '587 patent teaches a method for dispensing viscous fluid into the eye during surgery upon demand, without the need to re-insert a viscoelastic syringe or needle each time the viscoelastic materials are added. In particular, the '587 patent teaches using a dispensing means which includes, in part, a flexible diaphragm defining a first chamber filled with viscoelastic materials and a second chamber filled with pressurized air. The second chamber is connected to a phacoemulsification machine adapted for providing a constant controlled source of air pressure. The first chamber is connected to a conduit in the phacoemulsification handpiece which includes a means for dispensing the viscoelastic material to the eye and proximate the needle tip of the handpiece. The means for dispensing the viscoelastic material includes a normally closed valve disposed on the housing of the handpiece such that when the surgeon wants to inject additional viscoelastic material into the eye during surgery, he/she needs to open the valve. Upon opening the valve, the constant source of air pressure will push the viscoelastic material through the conduit into the handpiece whereby the material is injected into the eye proximate the needle tip.

Although the method disclosed in the '587 patent provides a method for dispensing viscoelastic materials without having to sporadically and repeatedly re-insert a needle into the eye, during surgery, it still requires and depends solely on the surgeon's ability to visually observe the amount of viscoelastic materials in the eye and the surgeons judgment on whether additional viscoelastic materials need to be injected, thereby being susceptible inaccuracy due to differences in visual capabilities and experiences from surgeon to surgeon.

Thus, there is a need for an improved system and: method for administering viscoelastic materials into the eye, wherein the decision as to the amount and timing of viscoelastic materials injected into the eye does not depend solely on the surgeon's visual capabilities and judgment.

### SUMMARY

It is to be understood that both the following summary and the following detailed description are exemplary and explanatory only and are not restrictive, as claimed. In one aspect, provided are methods and systems for dispensing viscoelastic materials into an eye during surgery without relying solely on the visual acuity and experience of the surgeon to determine if and when viscoelastic materials should be added. Instead, provided are systems and methods that automatically dispense viscoelastic materials into the eye based on a measurement of one or more Operating conditions or parameters such as intraocular pressure of the eye, injection speed, and injection volume.

In an aspect, the system comprises a controller, an intraocular pressure sensor, and a phacoemulsification handpiece, wherein the controller comprises a viscoelastic materials pump and a processor for activating and deactivating the viscoelastic materials pump based on a measurement of the intraocular pressure of the eye by the intraocular pressure sensor wherein, upon activation, the pump dispenses viscoelastic materials into the eye through the handpiece.

In an aspect, the system comprises a controller, an intraocular pressure sensor, a phacoemulsification handpiece, and a maintainer, wherein the controller comprises a viscoelastic materials pump and a processor for activating and deactivating the pump based on a measurement of the intraocular pressure of the eye by the intraocular pressure sensor wherein, upon activation, the pump dispenses viscoelastic materials into the eye through the maintainer.

In another aspect, the system further comprises a means for manually activating and deactivating the pump.

In another aspect, the intraocular pressure sensor comprises an inductance-capacitance (LC) resonant circuit implanted in the anterior chamber of the eye, and wherein the controller comprises an intraocular pressure detector for calculating a measurement of the intraocular pressure based on the resonant frequency of the LC resonant circuit.

In another aspect, the intraocular pressure sensor comprises a pressure-sensitive nanophotonic structure implanted into the anterior chamber of the eye, the pressure-sensitive nanophotonic structure having an optical signature that changes as a function of the intraocular pressure of the eye.

In another aspect, the intraocular pressure sensor comprises a programmable intraocular pressure sensor system implant integrated on a single CMOS chip, wherein the CMOS chip comprises a micromechanical pressure sensor (MEMS) array, a temperature sensor, an antenna, a capacitive powering array, readout and calibration electronics, a microchip-based digital control unit, and an RF-transponder.

In another aspect, a system comprises a viscoelastic material dispensing system having a mechanical device for injecting viscoelastic material through a cannula into the eye, and an electronic system for measuring the intraocular pressure of the eye, and the speed and volume of the viscoelastic material being injected by the mechanical device into the eye.

Additional embodiments and advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE FIGURES

The invention and the following detailed description of certain embodiments thereof may be understood with reference to the following figures:

FIG. 1 is a schematic diagram of an exemplary system in accordance with the invention showing a means for injecting viscoelastic materials into an eye through a phacoemulsification handpiece.

FIG. 2 is a schematic diagram of an exemplary system in accordance with the present invention showing a means for injecting viscoelastic materials into an eye through a maintainer.

FIG. 3 is a schematic diagram of an exemplary system in accordance with the invention showing an electronic system for controlling injection of viscoelastic materials into an eye by a mechanical injecting means, based on one or more measurements Of intraocular pressure, injection speed, and/or injection volume.

### DETAILED DESCRIPTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting, but to provide an understandable description of the invention.

The terms “a” or “an,” as used herein, are defined as one or more than one. The term “another,” as used herein, is defined as at least a second or more. Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes. In addition, this disclosure uses certain terms relating to exemplary phacoemulsification surgery devices and systems. For example, the terms “handpiece”, “controller”, “pump”, “probe”, “needle”, and “cannula” are used herein for convenience, and are not intended to limit the scope of the disclosure to a particular phacoemulsification device or system.

Disclosed are components that can be used to perform the described methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. The present methods and systems may also take the form of web-implemented computer software. Any suitable computer-readable storage medium may be utilized including hard disks, optical storage devices, or magnetic storage devices.

Embodiments of the methods and systems are described below with reference to block diagrams and flowchart illustrations of methods, systems, and apparatuses. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

As described in the background, a cataract is a clouding of the lens inside the eye. One procedure for cataract surgery is phacoemulsification. Phacoemulsification involves the breaking up of the cloudy lens into small pieces, which are then gently removed from the eye with suction. In phacoemulsification surgery, the surgeon uses a handpiece having an ultrasonically-driven horn and cutting tip. The handpiece is connected to a controller through an electrical cable. During the phacoemulsification procedure, the controller varies the ultrasonic power level transmitted by the handpiece to the horn and cutting tip for emulsifying the cloudy lens tissue. The handpiece is also connected to a flexible tube used to provide irrigation fluid to the eye. The handpiece is typically configured to dispense the irrigation fluid proximate the cutting tip. The handpiece is also connected to a flexible tube for aspirating the irrigation fluid and any emulsified eye tissue. The handpiece is typically configured aspirate through a separate sleeve inserted into the eye. Further, the handpiece is connected to a flexible tube for dispensing viscoelastic materials into the eye. The viscoelastic materials are used to protect sensitive eye tissue during the surgery. The handpiece is configured to inject the viscoelastic materials into the eye proximate the needle tip.

As described in the background, the viscoelastic material is typically inserted into the eye at the beginning of the surgery. However, during the surgery, the ultrasonic vibrations, irrigation and aspiration activities tend to wash away the viscoelastic materials, thereby leaving the sensitive eye tissue, e.g., endothelial cells less protected. As a result the surgeon typically needs to periodically add viscoelastic materials to the eye during the surgery.

As described in the background, the known methods for dispensing or injecting the viscoelastic materials into the eye require the surgeon to visually inspect the eye during surgery, observe the amount of viscoelastic material present in the eye, and make a judgment as to whether more viscoelastic material needs to be added. The additional viscoelastic material can then be added using a pump manually activated by the surgery or a syringe having a needle manually inserted into the eye. In either case, the addition of viscoelastic material to the eye depends solely on the visual inspection/observations and judgment of the surgeon. This not only distracts the surgeon from other aspects of the surgery including, for example, cutting and aspirating the eye tissue, it also tends to provide different levels of accuracy from surgeon to surgeon. Thus, there is a need for an improved system and method for administering viscoelastic materials into the eye that enables the surgeon to pay more attention to the cutting and emulsifying aspects of the surgery and does not rely solely on the surgeon's visual acuity and judgment.

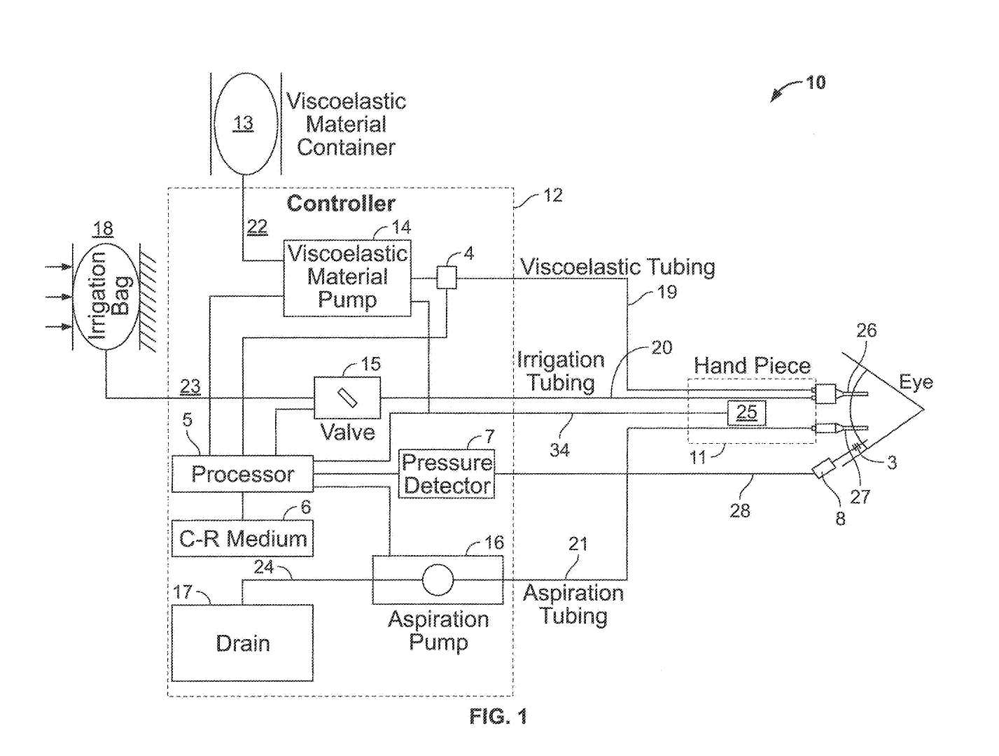

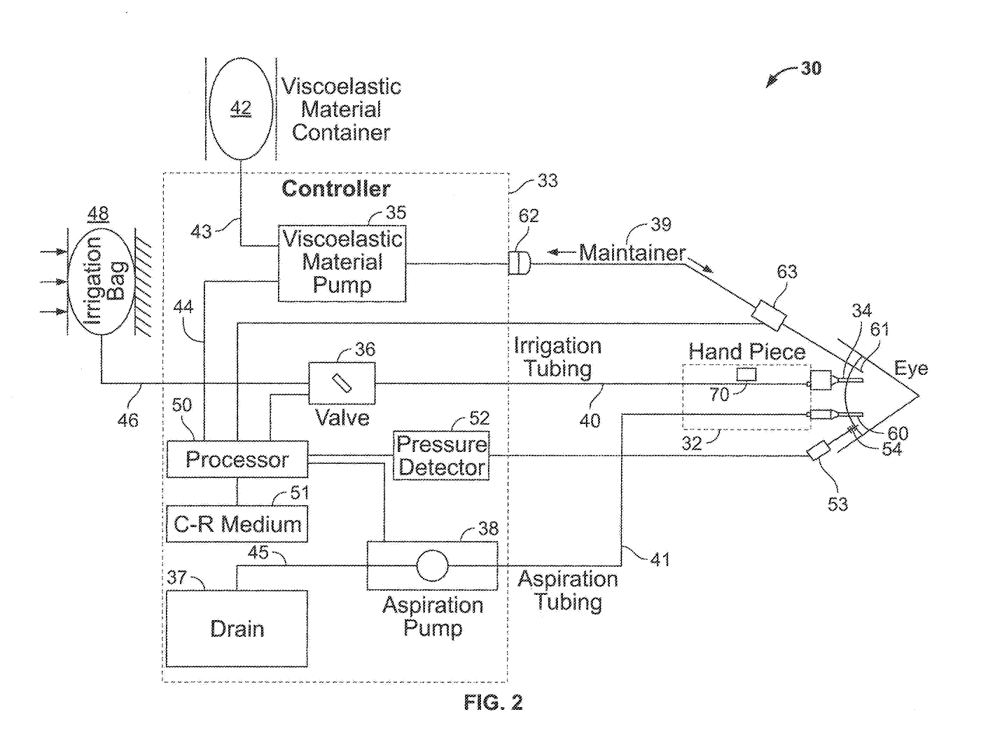

Referring now to FIGS. 1 and 2, embodiments of systems in accordance with the present invention are shown. For simplicity, the exemplary systems shown in FIGS. 1 and 2 are directed to systems and methods useful in ophthalmic surgical procedures, and are similar in structure and operation except that they comprise different means for dispensing or injecting viscoelastic materials into the eye. FIG. 1 shows an exemplary means for dispensing and injecting viscoelastic materials into an eye through a phacoemulsification handpiece, whereas FIG. 2 shows an exemplary means for dispensing and injecting viscoelastic materials into an eye through a maintainer. As will be explained in more detail below, various other structures, systems and methods are contemplated and thus it should be understood that the systems of FIGS. 1 and 2 are only exemplary embodiments of systems and methods in accordance with the present invention.

In FIG. 1, there is shown a system 10 comprising a controller 12 and a handpiece 11, wherein handpiece 11 is shown having a needle 26 and an aspiration tube 27, each inserted into an eye (not part of system 10). Needle 26 includes an ultrasonically-driven horn and a needle tip configured to cut eye tissue, and may be of any conventional suitable design heretofore used in phacoemulsification handpieces. Similarly, as in conventional phacoemulsification handpieces, handpiece 11 is electrically connected (not shown) to Controller 12 such that controller 12 can vary the ultrasonic power delivered by handset 11 through needle 26 for the emulsification of tissue in the eye. Thus, similar to conventional phacoemulsification handpieces, the tip of needle 26 upon vibrating at ultrasonic frequencies is capable of cutting or fragmenting eye tissue. Thus, needle 26 provides means for radiating ultrasonic energy into an eye in order to cut, fragment or emulsify tissue, depending on the particular surgical procedure being conducted.

Controller 12 comprises a processor 5 electrically connected to a computer-readable medium 6, an aspiration pump 16, a pressure detector 7, a valve 15, a viscoelastic material pump 14, and an electrical switch 25 on handpiece 11. Electrical switch 25 can be manually operated by a surgeon to switch between an on and an off state. When switch 25 is switched to the on state, a signal is transmitted to processor 5 to direct processor 5 to activate viscoelastic pump 14. When switch 25 is switched to the off state, a signal is transmitted to processor 5 to deactivate the viscoelastic pump 14.

Computer readable medium 6 stores programs for operating controller 12 including, but not limited to, a program for controlling the operation of viscoelastic material pump 14, a program for controlling the operation of aspiration pump 16, a program for controlling the operation of pressure detector 7, a program for controlling the operation of valve 15, a program for controlling the ultrasonic power delivered by handpiece 11 to needle 26, and a program for processing an electrical signal from electrical switch 25 on handpiece 11. Processor 5 can call and execute these programs from computer-readable medium 6, as needed.

Viscoelastic materials pump 14 is connected to a viscoelastic materials container 13 through a tube 22 and to handpiece 11 through viscoelastic tube 19. Viscoelastic materials container 13 can be any type of container suitable for holding and dispensing viscoelastic materials. Viscoelastic container 13 can be refillable or removable such that replacement viscoelastic materials can be added to container 13 or a replacement container can be attached to tube 22, as needed. Further, viscoelastic container 13 is configured such that when attached to tube 22 and upon activation of viscoelastic pump 14, viscoelastic material in container 13 can be drawn through tube 22 by to viscoelastic pump 14, and pumped to handpiece 11 through viscoelastic tube 19. Handpiece 11 can thereby inject the viscoelastic materials into the eye through needle 26. The viscoelastic material container 13 can contain any type of viscoelastic materials including, for example, Viscoat® and Healon®.

Valve 15 is connected to an irrigation bag 18 through a tube 23 and to handpiece 11 through an irrigation tube 20. Irrigation bag 18 can contain any type of irrigation fluid including, for example, a saline solution. Irrigation bag 18 is removable such that a replacement bag can be attached to tube 23, as needed. Irrigation bag 18 is configured such that when attached to tube 23 and upon the opening of valve 15, the irrigation fluid in irrigation bag 18 can be fed to through tube 23 to handpiece 11 and injected into the eye through needle 26.

Aspiration pump 16 is connected to handpiece 11 through aspiration tube 21, and to drain 17 through tube 24. Drain 17 can be any type of container for collecting aspirated fluids, materials and emulsified tissue from the eye. Upon activation of aspiration pump 16, fluids, materials and emulsified tissue in the eye can be drawn or vacuumed from the eye through sleeve 27 to aspiration tube 21 and fed to drain 17 through tube 24.

Pressure Detector 7 is electrically connected through electrical cable 28 to an intraocular pressure-monitoring system 8 coupled to an intraocular pressure sensor 3 (shown inserted into the eye). The intraocular pressure sensor 3 is operable to generate pressure measurement data related to the intraocular pressure of the eye. The pressure measurement data is captured and transmitted by pressure-monitoring system 8 to pressure detector 7 over communication cable 28. Pressure detector 7 along with processor 5 use the data to compute a measured intraocular pressure of the eye.

It should be appreciated therefore that, in system 10, the means for measuring the intraocular pressure of the eye comprises an intraocular pressure sensor 3, an intraocular pressure-monitoring system 8, a pressure detector 7 and a processor 5 communicating with a computer-readable medium 6. It should be understood, however, that the present invention is not limited to the means for measuring the intraocular pressure of system 10. A system and method in accordance with the invention can comprise any means for measuring the intraocular pressure comprising any type of intraocular pressure sensor and any type of pressure monitoring system know in the art.

For example, a means for measuring the intraocular pressure in a system in accordance with the present invention could comprise a processor, a pressure monitoring system and an intraocular pressure sensor comprising an inductance-capacitance (LC) resonant circuit, wherein the LC resonance circuit has a resonance frequency that changes as a function of changes in the intraocular pressure, wherein the intraocular pressure monitoring system comprises a coil for sensing the changes in the resonance frequency and a means for transmitting data, based on the measure changes in resonance frequency, to the processor, which calculates the measured intraocular pressure based on the data.

As another example, a means for measuring the intraocular pressure in a system in accordance with the present invention could comprise an intraocular pressure sensor comprising a pressure-sensitive nanophotonic structure, a pressure monitoring system comprising an optical reader, wherein the optical reader optically excites the nonophotonic structure and detects the reflected light, whose optical signature changes as a function of the intraocular pressure. The optical signature data can then be processed to determine the measured intraocular pressure.

In yet another example, a means for measuring the intraocular pressure in a system in accordance with the present invention could comprise a programmable intraocular pressure sensor system implant integrated on a single CMOS chip, wherein the CMOS chip comprises a micromechanical pressure sensor (MEMS) array (i.e., an intraocular pressure sensor), and a pressure monitoring system comprising an antenna, a capacitive powering array, readout and calibration electronics, a microchip-based digital control unit, and an RF-transponder, wherein the pressure monitoring system can wirelessly communicate the intraocular pressure reading (or data related thereto) to a remote processor including, for example, a phacoemulsification controller for receiving the intraocular pressure measurement or for determining the intraocular pressure measurement based on the data, as the case may be.

In all such embodiments, in accordance with the invention, the processing of the pressure sensor data captured by the pressure-monitoring system can be be performed by a processor located in intraocular pressure sensor, the pressure-monitoring system, or by a remote pressure detector and/or processor including, for example, a processor located in a controller for a phacoemulsification system.

System 10 can utilize the means for measuring the intraocular pressure to automatically dispense and/or inject viscoelastic materials into the eye during surgery. That is, in accordance with the invention, viscoelastic materials can be injected into the eye as a function of intraocular pressure. In operation, processor 5 calls the program for monitoring the intraocular pressure form computer-readable medium 6. During execution of the program, processor 5 directs pressure detector 7 to utilize intraocular pressure sensor 3 and pressure monitoring system 8 to obtain data related to the intraocular pressure of the eye. As described above, different methods for obtaining the data will be employed depending on the type and structure of intraocular pressure sensor 3 and pressure monitoring system 8. Once the data is returned to pressure detector 7, processor 5 can determine a measured intraocular pressure based on the data. The measured intraocular pressure level depends, in part, on the amount of viscoelastic material in the eye. The measured intraocular pressure is compared to a target pressure level, wherein the target pressure level is selected to be a level that provides for a desired amount of amount of viscoelastic material to be present in the eye. Thus, if the measured intraocular pressure is below the target level, processor 5 activates viscoelastic materials pump 14, unless viscoelastic material pump 14 is already activated. Upon and during activation, viscoelastic materials pump 14 draws viscoelastic materials from container 13 and pumps the materials to handpiece 11 through viscoelastic tube 19. Handpiece 11 thereby injects the viscoelastic materials into the eye through needle 26, to thereby increase the measured intraocular pressure of the eye to a level indicative a the appropriate amount of viscoelastic material in the eye to protect eye tissue during surgery. If the measured intraocular pressure level is at or above the target intraocular pressure level, the processor 5 deactivates the viscoelastic materials pump 14, unless the pump is already deactivated.

As a safeguard against the target pressure being set too low, or the means for measuring the intraocular pressure being inaccurate, system 10 provides for means to inject viscoelastic material on demand. This can be accomplished by manually operating switch 25 on handpiece 11. Upon detecting that switch 25 has been manually switched to the on state, processor 5 will activate viscoelastic pump 14, thereby providing viscoelastic materials to handpiece 11 in the same manner as described above. Upon detecting that switch 25 has been manually switched to the off state, processor 5 will deactivate viscoelastic pump 14, unless it is deter pined that that the intraocular pressure is below the target intraocular pressure level which, in that case, processor 5 would not deactivate viscoelastic pump 14.

It should be appreciated that the target intraocular pressure level can be determined and set in a number of different ways and based on any number of factors. For example, the surgeon can determine the target pressure level based on his/her experience, the conditions of the surgery, the characteristics of the eye under surgery, the age of the patient, the gender of the patient, the health of the patient, and the type of viscoelastic materials being used in the surgery.

In an embodiment, the surgeon could enter the target pressure level through a keyboard located on controller 12. In another embodiment, the surgeon could enter the target pressure level through an interface located on handset 11. In yet another embodiment, the surgeon could enter the target pressure level from a terminal or handheld device communicating with the controller 12 through a wireless interface. In yet another embodiment, the surgeon could enter the factors into controller 12, wherein controller 12 will calculate the target pressure level.

It should also be appreciated that the target pressure level can be adjusted or changed during surgery. Therefore, it is contemplated that, if the surgeon determines that the target pressure level is not causing the controller 12 to activate the viscoelastic pump 14 to provide enough viscoelastic material during surgery, the system 10 in an exemplary embodiment of the invention, will have means for enabling the surgeon to adjust or change the target level on demand. For example, in an embodiment, the handpiece 11 can have a set of buttons, one for increasing the target pressure value and the other for decreasing the target pressure value. The buttons can be electrically connected to controller 12 or alternatively, communicate with controller 12 through other means including, for example, a wireless interface. Controller 12 can then use the new or changed target pressure level when it runs the programs for comparing the measured intraocular pressure to the target pressure for controlling the operation of the viscoelastic material pump 14.

In another embodiment, the handpiece 11 can have an interface including, for example, a keyboard or touch screen for inputting changes to the value of the target pressure level. The changes can then be communicated to the controller 12 through an electrical connection (not shown) between the handpiece 11 and the controller 12 or by any other communication means including, for example, a wireless connection.

In another embodiment, the controller 12 can have an interface for inputting changes to the target pressure level. The interface could be, for example, a touch screen or a set of buttons, similar to that described above for embodiments of the handpiece 11.

By providing the means for automatically adding viscoelastic materials into the eye based on intraocular pressure, system 10 reduces the surgeon's distraction from other aspects of the surgery and provides for a more consistent and accurate handling of viscoelastic materials in the eye from surgeon to surgeon. In addition, by enabling the surgeon to request through switch 25 on handpiece 11, the injection of additional viscoelastic material into the eye, system 10 provides for addition protection and flexibility for making sure the sensitive tissue in the eye is well-protected during surgery.

In another embodiment, System 10 may also control the injection viscoelastic materials into the eye based on the injection speed and volume. That is, viscoelastic materials can be injected into the eye as a function of intraocular pressure, injection speed, injection volume, or some combination thereof. As shown in FIG. 1, System 10 has injection-measuring device 4 connected to viscoelastic tubing 19. It should be noted that although injection-measuring device 4 is shown as being located in controller 12, it can be located anywhere along the viscoelastic tubing 19 or anywhere near the point where the viscoelastic materials enter into the eye. Also, injection-measuring device 4 may utilize any means known by those skilled in art for measuring the volume and speed of fluids such as viscoelastic materials. In such an embodiment, controller 12 may be programmed to compare the measured injection speed and volume and compare to desired levels of injection speed and volume and, based on such comparison, can adjust the volume and speed of the injection of viscoelastic materials into the eye, including turning the viscoelastic pump on and off, to achieve the desired levels.

It should be understood that although system 10 shows viscoelastic material pump 14 integrated into controller 12, the present invention is not limited as such. It is contemplated that viscoelastic material pump 14 can be located external to controller 12. In such a case, viscoelastic material pump 14 can be controlled by controller 12 or a separate controller, wherein the separate controller can be a stand-alone device or the separate controller can be integrated into the viscoelastic pump or the handpiece 11. It is also contemplated that the viscoelastic material container 13 and the viscoelastic material pump 14 can be integrated into handpiece 11 while the viscoelastic material container 13 can also be removable therefrom for purposes of filling with viscoelastic materials or replacement. The viscoelastic, container 13 can be made of easily assembled components and materials such that it may be entirely disposable.

Similarly, although system 10 has a single handpiece 11, wherein the functions of cutting, irrigation, ultrasonic emulsifying, aspiration, and injecting viscoelastic materials are all integrated into one handpiece 11, the present invention is not limited as such. It is contemplated that such functions can be implemented through any number of handpieces. For example, it is contemplated that one handpiece can be configured to provide the cutting, irrigating, emulsifying, and aspirating, and a second handpiece can be configured to provide the injecting of viscoelastic materials.

Further, it is contemplated that the viscoelastic material can be dispensed into the eye without having to go through a handpiece at all. Instead, the viscoelastic material can be pumped through a tube such as, for example, a maintainer having a long braided tip inserted into an incision in the eye.

Referring now to FIG. 2, there is shown an exemplary system 30 comprising means for dispensing or injecting viscoelastic materials into an eye using a maintainer in accordance with the present invention. For simplicity, system 30 has a similar structure to system 10 shown in FIG. 1 and described, except for the means for dispensing or injecting viscoelastic materials into the eye. In system 10, the means for injecting viscoelastic materials into the eye comprised a processor 10 electrically connected to a viscoelastic pump 14, wherein the primp is connected to a viscoelastic materials container 13 and a phacoemulsification handpiece 11, wherein upon activation of the pump by processor 5, viscoelastic materials would be pumped from viscoelastic materials container 13 to handpiece 11, which would inject the viscoelastic material through needle 26.

In contrast, the means for delivering/injecting viscoelastic materials into the eye in system 30 comprises a processor 50 electrically connected to a viscoelastic materials pump 35, wherein pump 35 is connected to a viscoelastic materials container 42, and a maintainer 39, wherein container 39 has a first end connect to a port on a controller 33 containing the pump 35 and processor 50, and a second end comprising a long braided tip (shown inserted into the eye. As a result, when processor 50 activates pump 35, viscoelastic materials from viscoelastic materials container 42 are dispensed to maintainer 39 and injected into the eye through the long braided tip 61.

The following provides a more complete description of system 30. As shown in FIG. 2, system 30 comprises a controller 33, a handpiece 32, and a maintainer 39, wherein handpiece 32 is shown having a needle 34 and an aspiration tube 60, each inserted into an eye (not part of system 30). Needle 34 includes an ultrasonically-driven horn and a needle tip configured to cut eye tissue, and may be of any conventional suitable design heretofore used in phacoemulsification handpieces. Similarly, as in conventional phacoemulsification handpieces, handpiece 32 is electrically connected (not shown) to Controller 12 such that controller 12 can vary the ultrasonic power delivered by handset 32 through needle 34 for the emulsification of tissue in the eye. Thus, similar to conventional phacoemulsification handpieces, the tip of needle 34, upon vibrating at ultrasonic frequencies, is capable of cutting or fragmenting eye tissue. Thus, needle 34 provides means for radiating ultrasonic energy into an eye in order to cut, fragment or emulsify tissue, depending on the particular surgical procedure being conducted.

Controller 33 comprises a processor 50 electrically connected to a computer-readable medium 51, an aspiration pump 38, a pressure detector 52, a valve 36, a viscoelastic material pump 35, and an electrical switch 70 on handpiece 32. Processor 50 is operable to call programs stored on computer-readable medium 51. The programs stored on computer-readable medium 51 include, hut are not limited to, a program for controlling the operation of viscoelastic material pump 35, a program for controlling the operation of aspiration pump 38, a program for controlling the operation of pressure detector 52, a program for controlling the operation of valve 36, a program for controlling the ultrasonic power delivered by handpiece 32 to needle 34, and a program for processing an electrical signal from electrical switch 70 on handpiece 32. Processor 50 can call and execute these programs from computer-readable medium 51, as needed.

Viscoelastic materials pump 35 is connected to a viscoelastic materials container 42 through a tube 43 and to a maintainer 39. Viscoelastic materials container 42 can be any type of container suitable for holding and dispensing viscoelastic materials. Viscoelastic container 42 can be refillable or removable such that replacement viscoelastic materials can be added to container 42 or a replacement container can be attached to tube 43, as needed. Further, viscoelastic container 42 is configured such that when attached to tube 43 and upon activation of viscoelastic pump 35, viscoelastic material in container 42 can be drawn through tube 43 by viscoelastic pump 35, and pumped to maintainer 39. Maintainer 39 has one end 62 connected to a port on controller 33, and a long braided tip 61 at the other end. Long braided tip 61 is shown inserted into the eye. Long braided tip 61 and end 62 of maintainer 39 are connected by a tube configured for carrying viscoelastic materials therethrough. The viscoelastic material container 42 can contain any type of viscoelastic materials including, for example, Viscoat® and Healon®.

Valve 36 is connected to an irrigation bag 48 through a tube 46 and to handpiece 32 through an irrigation tube 40. Irrigation bag 48 can contain any type of irrigation fluid including, for example, a saline solution. Irrigation bag 48 is removable such that a replacement bag can be attached to tube 46, as needed. Irrigation hag 48 is configured such that when attached to tube 46 and upon the opening of valve 36, the irrigation fluid in irrigation bag 48 can be fed through tube 40 to handpiece 32 and injected into the eye through needle 34.

Aspiration pump 38 is connected to handpiece 32 through aspiration tube 41, and to drain 37 through tube 45. Drain 37 can be any type of container for collecting aspirated fluids, materials and emulsified tissue from the eye. Upon activation of aspiration pump 38, fluids, materials and emulsified tissue in the eye can be drawn or vacuumed from the eye through sleeve 60 to aspiration tube 41 and fed to drain 37 through tube 45.

Pressure Detector 52 is electrically connected through an electrical cable to an intraocular pressure-measuring device consisting of an intraocular pressure sensor 54 (shown inserted into the eye) and a transducer 53. The intraocular pressure sensor 54 is operable to measure the intraocular pressure of the eye and transducer 53 is operable to convert the measurement to an electric signal transmitted to pressure detector 52. The different types of devices and methods for measuring intraocular pressure using a sensor inserted into the eye are well know by those skilled in the art.

In operation, in accordance with the techniques disclosed herein, during surgery, viscoelastic materials are injected into the eye as a function of intraocular pressure. Processor 50 calls the program for monitoring the intraocular pressure form computer-readable medium 51. During execution of the program, processor 50 directs pressure detector 52 to utilize intraocular pressure sensor 54 and transducer 53 to obtain a measurement of the intraocular pressure of the eye. The measured intraocular pressure level is reported to processor 52, which compares the measured pressure level to a target pressure level. If the measured level is below the target level, processor 50 activates viscoelastic materials pump 35, unless viscoelastic material pump 35 is already activated. Upon and during activation, viscoelastic materials pump 35 draws viscoelastic materials from container 42 and pumps the materials to maintainer 39 which thereby injects the viscoelastic materials into the eye through the long braided tip 61. If the measured intraocular pressure level is at or above the target intraocular pressure level, the processor 50 deactivates the viscoelastic materials pump 35, unless the pump is already deactivated.

Viscoelastic materials can also be injected on demand by the surgeon. In the event that the surgeon wants to inject additional viscoelastic materials, even though the intraocular pressure may be at or above the target level, the surgeon can activate the viscoelastic pump by operating switch 70 on handpiece 32. Upon detecting that switch 25 has been manually switched to the on state, processor 50 will activate viscoelastic pump 35, thereby providing viscoelastic materials into the eye through maintainer 39 in the same manner as described above. Upon detecting that switch 70 has been manually switched to the off state, processor 50 will deactivate viscoelastic pump 35, unless it is determined that the measured intraocular pressure is below the target pressure level which, in that case, processor 50 would not deactivate viscoelastic pump 35.

It should be appreciated that an exemplary method in accordance with the present invention utilizes the herein described systems and includes in one instance the measuring of the intraocular pressure of the eye, comparing the measured intraocular pressure to a target pressure level and, if the measured intraocular pressure is below the target pressure level, automatically activating a pump for dispensing viscoelastic material into the eye, wherein the viscoelastic materials can be injected through a handpiece, a maintainer or by any other means.

In addition, the present invention may also include the steps of: (1) making an incision into the eye and introducing a phacoemulsification needle into the incision for cutting, fragmenting, and/or emulsifying eye tissue using a handpiece; (2) making a second incision into the eye and introducing an irrigation tube into the second incision for irrigating fluid and cut, fragmented and/or emulsified eye tissue from the eye; (3) introducing irrigation fluid proximate the needle into the eye; (4) measuring the intraocular pressure of the eye; (5) comparing the measured intraocular pressure to a target pressure level; (5) automatically dispensing viscoelastic material into the eye when the measured intraocular pressure is below the target pressure level.

The step of dispensing the viscoelastic materials can comprise the steps of activating a pump when the measure intraocular pressure is below the target pressure level, and deactivating the pump when the measured intraocular pressure is at or above the target pressure level.

It should be appreciated that in an alternate embodiment of system 30, in accordance with the present invention, viscoelastic material pump 35 can be a standard alone viscoelastic pump. In such an embodiment, viscoelastic materials pump 35 can be located external to controller 33 which would be operable to communicate with and/or control viscoelastic material pump 35 through any typo of communication medium including, by way of example, a wireless interface, a copper cable, or a combination of such medium. In operation, through such communication medium, processor 50 can communicate with or control the activation and deactivation of viscoelastic materials pump 35 as described above.

In another embodiment, System 30 may also control the injection viscoelastic materials into the eye based on the injection speed and volume. That is, viscoelastic materials can be injected into the eye as a function of intraocular pressure, injection speed, injection volume, or some combination thereof. As shown in FIG. 2, System 30 has injection-measuring device 63 connected to maintainer 39. It should be noted that although injection-measuring device 63 is shown as being located external to in controller 33, it can be located anywhere along the maintainer 39 or anywhere near the point where the viscoelastic materials enter the eye. Also, injection-measuring device 63 may utilize any means known by those skilled in art for measuring the volume and speed of fluids such as viscoelastic materials. In such an embodiment, controller 33 may be programmed to compare the measured injection speed and volume and compare to desired levels of injection speed and volume and, based on such comparison, can adjust the volume and speed of the injection of viscoelastic materials into the eye, including turning the viscoelastic pump on and off, to achieve the desired levels.

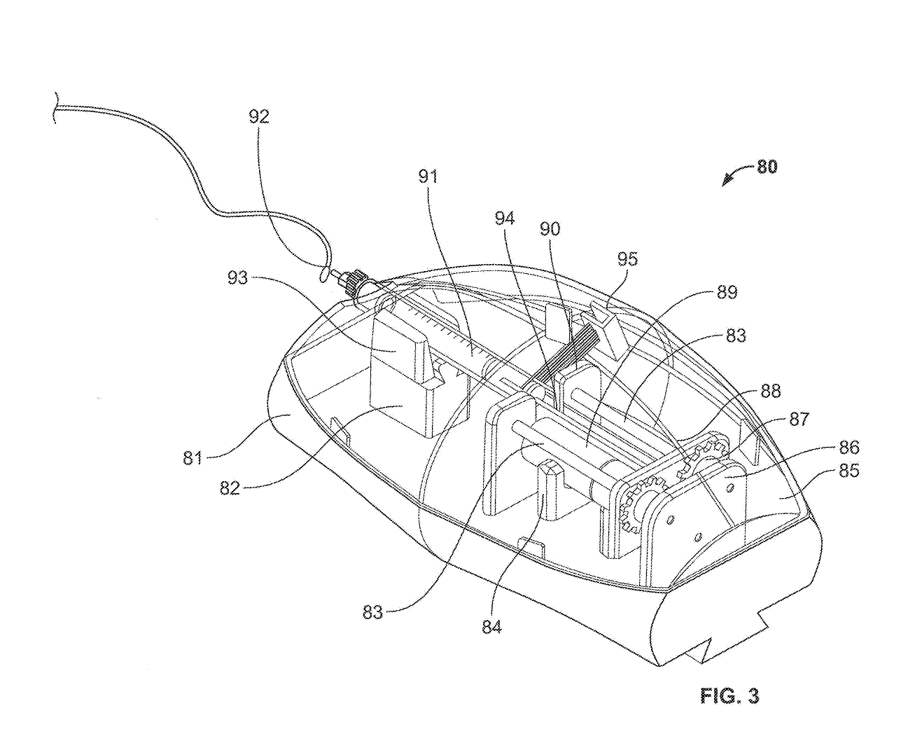

Referring now to FIG. 3, there is shown an exemplary embodiment of a stand-alone viscoelastic materials pump 80 in accordance with the present invention. As shown, viscoelastic materials pump 80 has a support base 81. Support base 81 is connected to and holds in place syringe guide holder 82, tab support 90, motor base 84, and gears support 86.

Syringe guide holder 82 has a top surface shaped to allow the attachment and removal of cylinder support 93, as desired. Cylinder support 93 has a curved top surface designed to support and hold in place syringe 91 when placed therein and to enable the removal of syringe 91 when desired. Syringe 91 has a cylindrical tube for containing a predetermined amount or volume of viscoelastic materials therein. Syringe 91 has a plunger 94 supported and held in place by tab support 90. Syringe 91 is connected to flexible cannula 92 and is operable to dispense viscoelastic materials contained in its cylindrical tube through flexible cannula 92 when depressed. Flexible cannula 92 may be fed into the eye for dispensing the viscoelastic materials into the eye during surgery.

Motor 89 is connected to and supported by motor base 84 which has a curved surface for holding motor 89 in place. Motor 89 is electrically connected to electrical interface 95 through which an external controller (not shown) can control the power and speed of the motor, and turn motor 89 on and off Motor 89 has a driving mechanism connected to gears 87 such that, when turned on, motor 89 can drive the gears 87 to rotate at a desired speed and power. Gears 87 are mechanically connected to threaded rods 83 which extend through fixed nuts (not shown) embedded in actuator 88. Actuator 88 is slide-able along threaded rods 88 and has a surface that can push against plunger 94 when it slides towards syringe 91.

In operation, when motor 89 is turned on, it will drive gears 87 to rotate threaded rods 83 to rotate along their axis in a counter-clockwise direction. The rotation of threaded rods 83 will mechanically interact with the nuts embedded in actuator 88 to drive actuator 88 to slide along the threaded rods and push on plunger 94. As plunger 94 is pushed it will force syringe 91 to dispense viscoelastic materials through flexible cannula 92 which may be fed into the eye during surgery. When motor 89 is turned off it will stop driving gears 87 and thereby stop any dispensing of viscoelastic materials into the eye.

Thus, it can be appreciated that motor 89 can be any motor operable to generate the mechanical power needed to provide the functionality of Viscoelastic materials pump 80 described herein. For example, motor 89 can be a DCX16L 12V motor.

It can also be appreciated that although viscoelastic materials pump 80 has been described in operation herein is connection with au external controller, viscoelastic materials pump 80, in another embodiment may contain its own controller that receives real-time measurements of intraocular pressure, and measurements of the speed and volume of viscoelastic materials being injected into the eye during surgery. Based on any one or more of these measurements, the controller may turn viscoelastic materials pump on and off to achieve the desired intraocular pressure, and speed and volume of viscoelastic materials being injected.

Further, it should be appreciated that a viscoelastic materials pump in accordance with the present invention is not limited to the embodiment shown and described far viscoelastic materials pump 80. In an alternate embodiment, a viscoelastic materials pump 80 may have multiple cylinder supports 93 for supporting multiple syringes 91, and a means for controlling and selecting which syringe 91 that actuator 88 engages to dispense viscoelastic materials into the eye.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but it is to be understood in the broadest sense allowable by law.

What is claimed is:

1. A system for delivering viscoelastic material to an eye, the system comprising:
An intraocular pressure detector for measuring the intraocular pressure of the eye; and
A means for automatically injecting viscoelastic material into the eye when the measured intraocular pressure is below a target pressure level.

2. The system of claim 1, wherein the intraocular pressure detector comprises an intraocular sensor and a transducer.

3. The system of claim 1 wherein, the intraocular pressure detector comprises an intraocular pressure monitoring system and a sensor comprising an inductance-capacitance (LC) resonant circuit, wherein the LC resonance circuit has a resonance frequency that changes as a function of changes in the intraocular pressure, wherein the intraocular pressure monitoring system comprises a coil for sensing the changes in the resonance frequency and a means for transmitting data, based on the measure changes in resonance frequency, to a processor, which calculates the measured intraocular pressure based on the data.

4. The system of claim 1 wherein the intraocular pressure detector comprise an intraocular pressure sensor comprising a pressure-sensitive nanophotonic structure, and a pressure monitoring system comprising an optical reader, wherein the optical reader optically excites the nonophotonic structure and detects reflected light, whose optical signature changes as a function of the intraocular pressure, thereby proving optical signature data for determining the measured intraocular pressure.

5. The system of claim 1 wherein the intraocular pressure detector comprises a programmable intraocular pressure sensor system implant integrated on a single CMOS chip, wherein the CMOS chip comprises a micromechanical pressure sensor (MEMS) array, a temperature sensor, an antenna, a capacitive powering array, readout and calibration electronics, a microchip-based digital control unit, and an RF-transponder.

6. The system of claim I further comprising a controller, a phacoemulsification handpiece, and a maintainer, wherein the controller is operable to activate and deactivating the means for automatically injecting viscoelastic materials into the eye based on a measurement of the intraocular pressure of the eye by the intraocular pressure detector wherein, upon activation, the pump dispenses viscoelastic materials into the eye through the maintainer.

7. The system of claim 1 further comprises a means for manually activating a pump to inject viscoelastic materials into the eye.

8. The system of claim 1, wherein the means for automatically injecting viscoelastic material comprises a pump for drawing viscoelastic materials from a container and for pumping the viscoelastic materials to a phacoemulsification handpiece, wherein the handpiece dispenses the viscoelastic materials into the eye through a needle inserted into the eye.

9. The system of claim 1 further comprising a means for measuring and controlling the volume and speed of the viscoelastic materials injected in the eye by said means for automatically injecting viscoelastic materials.

10. An ophthalmic surgical system comprising:
A handpiece, wherein the handpiece has a needle with a tip for radiating ultrasonic energy into an eye in order to cult, emulsify or fragment eye tissue;
An intraocular pressure detector for measuring the intraocular pressure of the eye;
A means for automatically injecting viscoelastic materials to the eye when the measured intraocular pressure of the eye is less than a target pressure level.

11. The system of claim 10, wherein the means for automatically injecting viscoelastic materials into the eye comprises a pump for pumping viscoelastic fluid from a container to the handpiece.

12. The system of claim 10, wherein the intraocular pressure detector comprises an intraocular sensor and a transducer.

13. The system of claim 10 wherein the intraocular pressure detector comprises an intraocular pressure monitoring system and a sensor comprising an inductance-capacitance (LC) resonant circuit, wherein the LC resonance circuit has a resonance frequency that changes as a function of changes in the intraocular pressure, wherein the intraocular pressure monitoring system comprises a coil for sensing the changes in the resonance frequency and a means for transmitting data, based on the measure changes in resonance frequency, to a processor, which calculates the measured intraocular pressure based on the data.

14. The system of claim 10 wherein the intraocular pressure detector comprise an intraocular pressure sensor comprising a pressure-sensitive nanophotonic structure, and a pressure monitoring system comprising an optical reader, wherein the optical reader optically excites the nonophotonic structure and detects reflected light, whose optical signature changes as a function of the intraocular pressure, thereby proving optical signature data for determining the measured intraocular pressure.

15. The system of claim 10 wherein the intraocular pressure detector comprises a programmable intraocular pressure sensor system implant integrated on a single CMOS chip, wherein the CMOS chip comprises a micromechanical pressure sensor (MEMS) array, a temperature sensor, an antenna, a capacitive powering array, readout and calibration electronics, a microchip-based digital control unit, and an RP-transponder.

16. The system of claim 10 further comprising a controller, and a maintainer, wherein the controller is operable to activate and deactivating the means for automatically injecting viscoelastic materials into the eye based on a measurement of the intraocular pressure of the eye by the intraocular pressure detector wherein, upon activation, the pump dispenses viscoelastic materials into the eye through the maintainer.

17. The system of claim 10 further comprising a means for manually activating and deactivating a pump to inject viscoelastic materials into the eye.

18. The system of claim 10, wherein the means for automatically injecting viscoelastic materials into the eye comprises a processor electrically connected to a pump and the intraocular pressure detector, wherein the processor is operable to activate the pump when the measured intraocular pressure is below a target pressure level and wherein, upon activation, the pump draws viscoelastic material from a container and feeds the viscoelastic material to a maintainer having a long braided tip inserted in the eye.

19. The system of claim 10 further comprising a means for measuring and controlling the volume and speed of the viscoelastic materials injected in the eye by said means for automatically injecting viscoelastic materials.

20. A method useful in ophthalmic surgical procedures, the method comprising the steps of:
Measuring the intraocular pressure of an eye;
Comparing the measured intraocular pressure to a target pressure level; and
Activating a pump for injecting viscoelastic material into the eye if the measured intraocular pressure is below the target pressure level.
